(19) United States
(12) Patent Application Publication
(10) Pub. No.: US 2014/0276048 A1
(43) Pub. Date: Sep. 18, 2014

(54) AUTOMATIC CONTINUOUS A-MODE ULTRASOUND MONITOR FOR PNEUMOTHORAX

(71) Applicant: UNIVERSITY OF FLORIDA RESEARCH FOUNDATION, INC., GAINESVILLE, FL (US)

(72) Inventors: SEAN P. KILEY, GAINESVILLE, FL (US); BARYS VALERIEVICH IHNATSENKA, GAINESVILLE, FL (US)

(73) Assignee: UNIVERSITY OF FLORIDA RESEARCH FOUNDATION, INC., GAINESVILLE, FL (US)

(21) Appl. No.: 14/212,604

(22) Filed: Mar. 14, 2014

Related U.S. Application Data
(60) Provisional application No. 61/791,933, filed on Mar. 15, 2013.

(51) Int. Cl. A61B 8/08; A61B 8/00

(52) U.S. Cl. CPC A61B 8/5223; A61B 8/08; A61B 8/4236; A61B 8/4483; A61B 8/56
USPC 600/438

(57) ABSTRACT

Automated continuous analysis of ultrasound data is provided to determine occurrence of pneumothorax in a patient. Specifically, A-mode ultrasound data is processed by a processing system to identify automatically if pneumothorax has occurred in a patient.

### CROSS-REFERENCE TO A RELATED APPLICATION

This application claims the benefit of U.S. provisional application Serial No. 61/791,933 filed Mar. 15, 2013, which is incorporated herein by reference in its entirety.

### BACKGROUND OF INVENTION

“Pneumothorax” refers to when air enters the pleural space between the lungs and the chest wall. In healthy individuals, this space is under negative pressure, so that the lungs can expand with the chest to breath. This pressure becomes positive when air enters the pleural space. This causes the lungs to collapse and can be life threatening. It is most often caused by blunt trauma to the chest, such as the trauma experienced in some car accidents. Pneumothorax can also develop as a complication following certain medical procedures, such as central venous catheter placement, mechanical ventilation, nerve blockade, etc.

Treatment of pneumothorax depends on the size of pneumothorax and its dynamic. Large growing pneumothorax is life threatening and usually a chest tube is inserted to evacuate air. The small pneumothorax could resolve spontaneously and only need observation. In certain instances, complications may arise from the treatment of pneumothorax, including death.

The dilemma with pneumothorax is that it can be difficult to diagnose and difficult to monitor, particularly pneumothorax progression. Except for visualization of gross violation of the chest cavity, palpation of chest wall crepitance (from pleural air entering the chest subcutaneous tissue), palpation of flail chest wall segments, or visualization of tracheal deviation, the physical exam is extraordinarily limited in its ability to diagnose most pneumothoraces. Auscultation of the chest to identify absent or diminished breath sounds and/or percussion of the chest to detect differences in conduction of mechanical energy are notoriously inaccurate even at sophisticated trauma centers.

The most accurate conventional method to determine if pneumothorax is present is to use either an x-ray or CT scan. Those techniques require time, money, educated personnel, and patient exposure to harmful radiation. Moreover, those techniques are not practical, especially where constant monitoring is necessary and the patient is not mobile (e.g., in the intensive care unit or the operating room). While ultrasound imaging systems have been used in the diagnosis of pneumotharaces, these systems are not portable or readily portable, nor are they able to automatically and continuously monitor for pneumotharaces. Further, conventional ultrasound imaging systems require significant clinical training and expertise to perform and properly interpret images to diagnose the pneumothorax.

Accordingly, a reliable, non-invasive monitor for continuous pneumothorax monitoring is needed.

### BRIEF SUMMARY

The present invention provides a system and method for continuously monitoring a patient for the development of a pneumothorax or for the progression of a pneumothorax in the patient. The present invention pertains to a system comprising a wearable patch that includes at least one ultrasound sensor (also referred to herein as an “ultrasound patch”) and a processing system configured to continuously receive and monitor signals generated by the ultrasound sensor(s). The subject ultrasound patch is adapted to be placed on the surface of the patient's chest for producing time domain “signals” that are analyzed to diagnose a pneumothorax. The ultrasound sensor(s) preferably includes at least one A-mode ultrasound transducer and at least one receiver, which produces one-dimensional, time domain signals to be analyzed by the processing system in A-mode ultrasound.

The subject invention automatically and continuously monitors the development and/or progress of pneumothorax in a patient in a reliable, accurate and particularly simple fashion.

### BRIEF DESCRIPTION OF THE DRAWINGS

FIG. 1 is a flowchart of the steps performed by the processing system that is configured to monitor ultrasound signals.

FIG. 2 shows one embodiment of a pneumothorax monitoring system of the invention.

FIG. 3 shows another embodiment of a pneumothorax monitoring system of the invention.

FIG. 4 is a cross section depiction of an embodiment of the invention as it is applied to the chest of a patient.

FIG. 5 is a depiction of a reference A-mode image of an ultrasound return signal for a non-pneumothorax patient.

FIG. 6 is a depiction of an A-mode image of an ultrasound return signal where pneumothorax is present.

### DETAILED DISCLOSURE

The present invention provides a method and apparatus for continuously monitoring a patient for the development of a pneumothorax. A pneumothorax monitoring system is provided comprising a wearable patch (also referred to herein as an “ultrasound patch”), wherein the patch comprises an ultrasound sensor, and a processing system that is configured to continuously receive and analyze signals from the ultrasound sensor(s) and, when necessary, provide a signal regarding the presence of pneumothorax. The ultrasound sensor is preferably an A-mode ultrasound sensor. The subject ultrasound patch is adapted to be placed on the surface of the patient's chest where air will most likely accumulate in case of pneumothorax development for producing time domain “signals” that are analyzed by the processing system to diagnose a pneumothorax.

According to an embodiment of the present invention, an A-mode ultrasound sensor is associated with a patch for placement on the surface of a patient's body, preferably along the bilateral parasternal borders of the patient's chest. Preferably, the patch includes an adhesive to permit retention of the patch in proper position on the patient's skin. In certain embodiments, the patch may further include a coupling medium, such as water, acoustic scanning gel, or mineral oil to assist in conducting the ultrasound signals.

An A-mode ultrasound sensor produces one-dimensional, time domain signals. The A-mode sensor comprises at least one transducer that generates an ultrasound signal and a receiver that receives the echoes from that signal as it goes through the anatomical structures that are beneath the transducer(s). The returning echoes from the signals produced by the transducer are represented as spikes (A-mode ultrasound signals). The height of and distance between spikes represent the amplitude and time, respectively, of the echoes and are proportional to the difference in acoustic impedances of the materials which form the interface from which the echo arose and to the depth of those structures beneath the transducer. Air in the pleural cavity that accumulates with pneumothorax will block the ultrasound signal so that the echoes received will be different from the echoes received from normal lungs/chest cavity without pneumothorax.

The A-mode ultrasound sensor comprises at least one transducer. In one embodiment, the A-mode ultrasound sensor comprises one transducer. In other embodiments, the A-mode ultrasound sensor comprises more than one transducer to increase the sensitivity and selectivity of the A-mode ultrasound signals. Preferably, the A-mode ultrasound sensor comprises about 2-10 transducers. Even more preferably, the A-mode ultrasound sensor comprise about 3-4 transducers.

A transducer of the A-mode ultrasound sensor converts electrical energy to mechanical energy to produce ultrasound waves as well as vice versa to convert mechanical energy (reflected sound waves) to electrical energy. A transducer comprises a piezoelectric material, which can be natural or synthetic. Examples of natural piezoelectric materials include, but are not limited to, crystals such as berlinite, topaz, gallium phosphate, quartz, and tourmaline. Examples of synthetic piezoelectric materials include, but are not limited to, barium titanate, lead titanate, lead zirconate titanate, lithium titanate, potassium niobate, sodium potassium niobate, lithium niobate, sodium tungstate, bismuth ferrite, bismuth titanate, and zinc oxide.

The penetration depth of the ultrasound wave is dependent on the frequency of the signal. To monitor pneumothorax development and/or progression, the transducer(s) can generate a signal at about 1 MHz-10 MHz frequency. More preferably, the transducer(s) can generate a signal at about 3 MHz-7 MHz frequency. Even more preferably, the transducer(s) can generate a signal at about 5 MHz frequency.

B-mode ultrasound imaging produces a two-dimensional image such as that provided on a video display, where each displayed line of the image corresponds to a single A-mode signal acquired from adjacent positions. The brightness or intensity of each pixel or dot along that line corresponds to the amplitude of received echoes. Unfortunately, B-mode transducers and units that are currently available are expensive, require a high degree of training to operate and cannot be used for continuous monitoring because the transducers are hand-held devices.

The pneumothorax monitoring system comprises a processing system configured to (a) continuously monitor and assess the ultrasound return signals, (b) identify return ultrasound signals indicative of pneumothorax, and (c) output a signal that indicates that pneumothorax was detected. The ultrasound return signals are preferably A-mode signals generated by the ultrasound sensor on the ultrasound path.

The processing system comprises a processing unit, such as microcontrollers, neural networks, parallel distributed processing systems, neuromorphic systems, or the like. Preferably, the processing unit is a microprocessor. The microprocessor may be analog or digital and should contain circuits to be programmed for performing the functions described herein, such as analysis of A-mode signals generated by the ultrasound patch to determine whether pneumothorax is present and/or progressing. Circuits or programs for performing these functions are conventional and well known. A microprocessor is preferred over dedicated analog or digital processors because it has the flexibility to be programmed to store and analyze data.

The processing system comprises an alarm device for communicating to a user a signal that indicates that pneumothorax was detected and/or progressing. The alarm device provides an audio and/or visual display indicating detection of and/or progression of pneumothorax.

The processing system can further include a data and/or memory storage for storing the return A-mode ultrasound signals.

In certain embodiments, the processing system further comprises a visual display and/or user interface. The processing system can further include the ability to analyze the return A-mode ultrasound signals and provide either A-mode and/or M-mode images of the return ultrasound signals for the user to review following communicating the signal to the user that pneumothorax was detected and/or progressing.

In certain embodiments, communication of return ultrasound signals from the ultrasound patch to the processing system is performed over a direct-wired connection. In other embodiments, communication between the ultrasound patch and the processing system is conducted over a wireless connection, such as via the use of wireless media such as acoustic, RF, infrared and other wireless media.

The processing system can be provided on the ultrasound patch. In other embodiments, the processing system is a separate unit from that of the ultrasound patch.

In one embodiment, the processing system is located within a ventilator monitor, wherein the processing system has instructions to (a) process the ultrasound return signals, (b) identify return ultrasound signals indicative of pneumothorax, and (c) output an indication that pneumothorax was detected. The ventilator monitor would have the ability to provide notice to the user that a pneumothorax has been detected.

In one embodiment, the processing system is configured to digitize the ultrasound signals and store them in memory. The stored data can be transmitted periodically or at a later time to the user. This delayed transmission may, without restriction, be utilized to improve battery life by transmitting data transiently, instead of continuously; or to allow for patient monitoring during disconnection from the ventilator monitor.

The subject invention also provides a method for continuously monitoring a patient for pneumothorax. The method includes the steps of: providing a pneumothorax monitoring system as described above; applying an ultrasound patch of the pneumothorax monitoring system to a patient, wherein the ultrasound patch comprises an ultrasound sensor configured to send and receive ultrasound return signals reflected from a target region in the patient's lungs; and activating the processing system to: monitor the ultrasound return signals; analyze the ultrasound return signals to identify return ultrasound signals indicative of pneumothorax, and, when signals indicative of pneumothorax are detected, output a signal indicating that pneumothorax was detected.

In certain embodiments, when pneumothorax is detected, the processing system communicates to the user the optimal treatment(s) for pneumothorax.

The step of analyzing the ultrasound return signals includes comparing the ultrasound return signals to an ultrasound signal as a reference for non-pneumothorax. When the result of the comparison indicates a change of the position or shape of the received ultrasound signal with respect to the reference ultrasound signal position or shape, a signal is transmitted to the user.

In certain embodiments, the processing system analyzes the ultrasound return signals and converts them into A-mode signals. In others, the receiver provides the ultrasound return signals as A-mode signals.

In an embodiment, a model, such as a neural network, is pretrained with clinical data and the input parameters regarding A-mode ultrasound signals from patients with or without pneumothorax. Once a model having a desired degree of predictability has been achieved and verified, the network output, such as an A-mode signal indicative of non-pneumothorax, may be used as an ultrasound signal reference.

The method comprises applying an ultrasound patch to the chest of a patient. More preferably, the ultrasound patch would be applied along the parasternal borders bilaterally.

FIG. 1 is a flowchart of the steps performed by the processing system that is configured to monitor ultrasound signals. The processing system is configured to receive and monitor ultrasound signals 10. The processing system will monitor the ultrasound signals to determine if there are any changes to the signals 20. Should there be a change in the ultrasound signals, the processing system will provide a signal indicating that a change in ultrasound signals was detected 30.

In one embodiment, the processing system would initiate monitoring the patient, wherein the patient does not have a pneumothorax, thus providing a baseline from which the ultrasound signals would be compared. In another embodiment, the processing system is provided with a baseline for the ultrasound signals to be monitored. Any changes from the baseline ultrasound signals would cause the processing system to provide a signal indicating that a change in the signals was detected.

Following is an example that illustrates a system and method for practicing the invention. This example should not be construed as limiting.

### EXAMPLE 1

FIGS. 2 and 3 are illustrations a pneumothorax monitoring system 40 of the invention. An ultrasound patch 50 is provided. The ultrasound patch includes an ultrasound sensor 60. The ultrasound patch 50 may be directly wired to a processing system 70 (FIG. 2). Alternatively, the ultrasound patch 50 includes wireless media 75 for communicating with the processing system 70 (FIG. 3).

FIG. 4 is a cross section depiction of the ultrasound patch 50 as it is applied to the chest of a patient. Specifically, the ultrasound sensor 60 comprises transducers 80 and a receiver 85 for generating A-mode ultrasound return signals to be monitored and analyzed by the processing system 70. The ultrasound patch 50 is applied to the skin 90 of the patient. The signals generated by the ultrasound sensor 60 will be representative of the ultrasound that travels through the skin 90, chest wall 95, parietal pleura 100, visceral pleura 105 and lung 110.

The system 40 would monitor the detection of any pneumothorax based on any changes to the ultrasound signals. The ultrasound signal is reflected by an interface between media with different acoustic impedance. In normal or baseline conditions, as illustrated in FIG. 5, ultrasound signals would be received regarding the pleura 120 and lung 125.

FIG. 6 illustrates an A-mode image of an ultrasound where pneumothorax is present. With pneumothorax (air) 115, modified or lack of ultrasound signals 130 would be received because no acoustic mismatch would be reflected, due to dissipation by air.

Once a pneumothorax 115 is detected, its extension and progress can be monitored with the system. Air in pleural cavity tends to accumulate near bilateral parasternal borders of the patient's chest, but as pneumothorax progresses, it tends to move everywhere. In certain instances, some pneumothorax will resolve spontaneously. In other instances, the pneumothorax can progress to tension pneumothorax that could be lethal. With the system, instead of placing a chest tube, which is a painful, invasive procedure with known complications, the ultrasound patch would be applied to an area away from sternum where the lung is still in contact with the chest wall (no disruption/change in signal) to determine if pneumothorax is present (e.g., progressing). If pneumothorax is detected, a signal can be provided to the user by the processing system 70, as well as notice that the pneumothorax is progressing and getting bigger. If pneumothorax is not detected, no further action would be needed and placement of a chest tube would be averted.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for continuously monitoring a patient for the development of a pneumothorax comprising:
providing a pneumothorax monitoring system comprising an ultrasound patch and a processing system, wherein the ultrasound patch comprises an A-mode ultrasound sensor, and wherein the processing system is configured to receive and analyze ultrasound return signals generated by the ultrasound sensor;
applying the ultrasound patch to a patient's chest; and
activating the processing system to analyze the ultrasound return signals, identify ultrasound return signals indicative of pneumothorax, and
when pneumothorax is identified, communicating an indication that pneumothorax was detected.

2. The method of claim 1, further comprising the step of communicating via the processing system a treatment for the pneumothorax.

3. The method of claim 1, wherein the ultrasound patch is applied on the chest along the parasternal borders bilaterally.

4. A system for continuously monitoring a patient for the development of and/or progression of a pneumothorax, comprising: an ultrasound patch, wherein the ultrasound patch comprises an A-mode ultrasound sensor; and a processing system.

5. The system of claim 4, wherein the ultrasound sensor comprises one transducer.

6. The system of claim 4, wherein the ultrasound sensor comprises more than one transducer.

7. The system of claim 4, wherein the ultrasound sensor on the ultrasound patch communicates with the processing system via a wire.

8. The system of claim 4, wherein the ultrasound sensor on the ultrasound patch communicates with the processing system via wireless media.

9. The system of claim 4, wherein the processing system is provided on the ultrasound patch.

10. The system of claim 4, wherein the processing system is a separate unit from the ultrasound patch.
